(19) United States
(12) Patent Application Publication
(10) Pub. No.: US 2013/0236480 A1
(43) Pub. Date: Sep. 12, 2013

(54) TRANSGLUTAMINASE 2 INHIBITORS FOR USE IN THE PREVENTION OR TREATMENT OF RAPIDLY PROGRESSIVE GLOMERULONEPHRITIS

(71) Applicants: Pierre-Louis Tharaux, Paris (FR); Cécile Fligny, Paris (FR)

(72) Inventors: Pierre-Louis Tharaux, Paris (FR); Cécile Fligny, Paris (FR)

(21) Appl. No.: 13/700,464

(22) PCT Filed: Jun. 1, 2011
(86) PCT No.: PCT/EP2011/059100

§ 371 Date: Feb. 15, 2013

(30) Foreign Application Priority Data
Jun. 2, 2010 (EP) 10305581.0

(51) Int. Cl. A61K 31/713; A61K 31/7105; A61K 45/06; A61K 31/18

(52) U.S. Cl. CPC A61K 31/713; A61K 31/18; A61K 31/7105; A61K 45/06
USPC 424/184.1; 514/603; 514/44 A; 514/44 R

(57) ABSTRACT

The present invention relates to TG2 inhibitors for use in the prevention or treatment of rapidly progressive glomerulonephritis and to pharmaceutical compositions thereof.

### FIELD OF THE INVENTION

The present invention relates to transglutaminase 2 (TG2) inhibitors for use in the prevention or treatment of rapidly progressive glomerulonephritis.

### BACKGROUND OF THE INVENTION

Glomerulonephritis (GN) refers to a heterogeneous group of diseases characterized by inflammatory changes in glomerular capillaries and accompanying signs and symptoms of an acute nephritic syndrome.

Among diseases of this group, Rapidly Progressive GlomeruloNephritis (RPGN), also called crescentic glomerulonephritis or extracapillary glomerulonephritis, consists of the most severe class of glomerulopathies in humans. This disease is a clinical syndrome and a morphological expression of severe glomerular injury. Glomerular injury manifests as a proliferative histological pattern, accumulation of T cells and macrophages, proliferation of intrinsic glomerular cells, accumulation of cells in Bowman's space (“crescents”), and rapid deterioration of renal function. RPGN usually presents with acute nephritic syndrome (acute renal failure (ARF), fall in urinary output, haematuria, proteinuria, cellular casts in the urine).

The causative agents in most forms of human glomerulonephritis are unknown, but some evidences show that glomerulonephritis follow bacterial or viral infections. Most evidence now suggests that infectious agents, and doubtless other stimuli as well, induce glomerulonephritis by triggering an autoimmune response that results in formation of immune-complex deposits in glomeruli or elicits a cell-mediated immune response to antigens in, or of, the glomerulus. Causes include Goodpasture syndrome and immune complex diseases such as systemic lupus erythematosus (SLE), acute proliferative glomerulonephritis, chronic infection with RPGN, cryoglobulinaemia, Henoch-Schönlein purpura, and IgA nephropathy. Also, anti-neutrophil cytoplasmic antibodies (ANCA)-associated vasculitis such as Wegener granulomatosis, microscopic polyangiitis, or Churg-Strauss syndrome or Wegener granulomatosis cause RPGNs. At last, some RPGN remain without known cause (idiopathic RPGN).

Available therapies for RPGN are limited to potent immunosuppressive drugs (high doses of cortico-steroids with cyclophosphamide that can be substituted by azathioprine after remission). In some cases, apheresis therapy is considered in patients with an aggressive form. Approximately one-fourth to one-third of patients experience a recurrence within several years. The need for maintenance immunosuppression therapy raises the problem of adequate immunosuppression versus toxicity. These different immunosuppressive regimens are associated with significant risk of opportunistic infection and sever metabolic side effects (mainly diabetes, dyslipidemia and osteoporosis). On the other hand, some categories of immune complex glomerulonephritis do not necessarily warrant extensive immunosuppressive therapy unless numerous active crescents are present. The prognosis is poor; at least 80% of people develop end-stage kidney failure within six months without treatment. The prognosis is better for people younger than 60 years or if an underlying disorder causing the glomerulonephritis responds to treatment. Surviving patients have an altered quality of life with requirement for side effects-prone non specific immune-suppressants and costly dialysis maintenance.

Thus, there is a need for novel therapies. By defining the conditions and requirements for disease initiation and progression, more specific and directed therapies could be developed for treatment and possibly prevention, which would represent a considerable improvement on current treatment protocols. New treatment permitting the decrease of doses of immunosuppression therapy is of great interest for limiting side effects of such therapy and improving the prognosis.

Tissue transglutaminase (transglutaminase 2, TG2) is a widely expressed enzyme that mediates post-translational protein modification and protein-protein interactions, mainly via cross-linking glutamine residues. TG2 is expressed in normal kidneys, at the glomerular and interstitial level, and is induced in kidney diseases, in human and animal. It is also expressed by the monocytes/macrophages, the T-cells and the neutrophils, which represent key effectors of this disease.

### SUMMARY OF THE INVENTION

The present invention relates to a TG2 inhibitor for use in the prevention or treatment of rapidly progressive glomerulonephritis (RPGN).

Particularly, the invention relates to said TG2 inhibitor in combination with an immunosuppressive treatment.

The invention also provides pharmaceutical compositions comprising a TG2 inhibitor of the invention.

### DETAILED DESCRIPTION OF THE INVENTION

The inventors showed marked podocytic expression of TG2 in mice and human kidneys in several conditions. Thus, they investigated the functions of this enzyme in severe podocytopathies such experimental rapidly progressive glomerulonephritis (RPGN).

Then, they found that TG2 is markedly expressed in crescentic lesions in mice and human glomerulonephritic subjects. They showed that this enzyme induces a migratory phenotype in podocytes in vitro and that TG2 deficiency protects mice from the development of renal failure in an experimental model of RPGN.

These results suggest that inhibiting the TG2 pathway would be clinically beneficial for the treatment of severe RPGN.

### TG2 Inhibitors

Thus, a first object of the invention relates to a TG2 inhibitor for use in the prevention or treatment of rapidly progressive glomerulonephritis (RPGN).

According to the invention, the term “TG2” has its general meaning in the art and refers to the tissue transglutaminase, abbreviated tTG or TG2. The TG2 protein can be from any source, but typically is a mammalian (e.g., human and non-human primate) TG2, particularly a human TG2.

The term “Rapidly Progressive Glomerulonephritis” (or RPGN) encompasses crescentic glomerulonephritis and extracapillary glomerulonephritis. Said RPGN is often caused or associated with several diseases such as Goodpasture syndrome, Immune complex diseases such as systemic lupus erythematosus, acute proliferative glomerulonephritis, chronic infection with RPGN, Henoch-Schönlein purpura, and IgA nephropathy, Anti-neutrophil cytoplasmic antibodies (ANCA)-associated vasculitis such as Wegener granulomatosis, microscopic polyangiitis, or Churg-Strauss syndrome or Wegener granulomatosis, but it can also be an idiopathic RPGN.

Thus, in a particular embodiment, the invention relates to a TG2 inhibitor for use in the prevention or treatment of RPGN associated to a disease selected from the group of Goodpasture syndrome, Immune complex diseases such as systemic lupus erythematosus, acute proliferative glomerulonephritis, chronic infection with RPGN, Henoch-Schönlein purpura, and IgA nephropathy, Anti-neutrophil cytoplasmic antibodies (ANCA)-associated vasculitis such as Wegener granulomatosis, microscopic polyangiitis, or Churg-Strauss syndrome or Wegener granulomatosis

The term “treating” or “treatment” refers to reversing, alleviating, inhibiting the progress of, or preventing the disorder or condition to which such term applies, or one or more symptoms of such disorder or condition.

The term “preventing” or “prevention” refers to preventing the onset of RPGN in a subject at risk of RPGN, particularly a subject afflicted with a disease causing RPGN (described above).

The term “TG2 inhibitor” should be understood broadly and encompasses any substance able to prevent the action of TG2, and more particularly to prevent the action of TG2 on podocytes, and more particularly for alleviating podocytes proliferation and/or migration.

Thus, one object of the invention relates to a tissue transglutaminase (TG2) inhibitor for use in a method for alleviating podocytes proliferation and/or migration in a subject afflicted with rapidly progressive glomerulonephritis (RPGN).

In one embodiment, an inhibitor of TG2 activity can be used.

The term “inhibitor of TG2 activity” should be understood broadly and encompasses substances acting directly on TG2 and able to prevent the interaction or binding between TG2 and its ligands.

So, said inhibitor of TG2 activity particularly encompasses classical inhibitors of TG2 activity which are small organic molecules well known in the art.

In one embodiment, an inhibitor of TG2 activity that could be used can be selected from the group of competitive amine inhibitors (Lorand et al, 1984). Some of the most commonly used competitive amine inhibitors, including putrescine, monodansylcadaverine (MDC), 5-(biotinamido) pentylamine, cystamine, spermidine, histamine and fluorescin cadaverine.

In another embodiment, an inhibitor of TG2 activity of the invention is selected from the group of reversible TG2 inhibitors. Examples of reversible TG2 inhibitors are GTP and GDP, the divalent metal ion Zn2+ or GTP analogues such as GTPγS and GMP-PCP (Lai et al., 1998; Lorand et al., 1984; Aeschlimann et al., 1994).

In a further embodiment, an inhibitor of TG2 activity of the invention can be selected from the group of irreversible TG2 inhibitors such as iodoacetamide (Folk et al., 1966; de Macedo et al., 2000), 3-halo-4,5-dihydroisoxazoles compounds, gluten peptides or 2-[(2-oxopropyl)thio]imidazolium derivatives (Freund et al., 1994; Hausch et al., 2003, Maiuri et al., 2005), or Cbz-gln-gly analogues derived from TG2 substrate carbobenzyloxy-Lglutaminylglycine (Cbz-gln-gly) as the inhibitor backbone (de Macedo et al., 2002; Pardin et al., 2006). In a further embodiment, an inhibitor of TG2 activity can be selected from peptidomimetic irreversible inhibitors using a gluten peptide sequence as the inhibitor backbone (Hausch et al., 2003).

In a further embodiment, an inhibitor of TG2 activity of the invention can be selected from substitued 3,4-dihydrothieno[2,3-d]pyrmidines (WO 2006060702) such as thieno[2,3-d]pyrimidin-4-one acylhydrazide (Duval et al, 2005; Case et al, 2005) and from substituted cinnamoyl benzotriazolyl amides and the 3-(substituted cinnamoyl)pyridines (Pardin et al., 2008).

In a particular embodiment of the invention, said inhibitor of TG2 activity is monodansylcadaverine.

An inhibitor of TG2 activity may be an antibody or antibody fragment that can partially or completely blocks the action of TG2 on its substrates (or the interaction between TG2 and its substrates).

In particular, the inhibitor of TG2 activity may consist in an antibody directed against TG2, in such a way that said antibody blocks the binding of TG2 on its substrates. More particularly, said antibody can be directed against the active site of TG2.

Antibodies directed against TG2 can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies of the invention can be prepared and isolated using any technique that provides for the production of antibody molecules by continuous cell lines in culture. Techniques for production and isolation include but are not limited to the hybridoma technique originally described by Kohler and Milstein; the human B-cell hybridoma technique and the EBV-hybridoma technique. Alternatively, techniques described for the production of single chain antibodies (see, e.g., U.S. Pat. No. 4,946,778) can be adapted to produce single chain antibodies of the invention. Inhibitors of TG2 activity useful in practicing the present invention also include antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity.

Humanized antibodies and antibody fragments thereof can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

According to the invention, the 6B9 antibody, that recognizes TG2 (Mohan et al, 2003), can be used.

Furthermore, the inhibitor of TG2 activity may be an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S D, 1999. Peptide aptamers consists of a conformationally constrained antibody variable region displayed by a platform protein, such as E. coli Thioredoxin A that are selected from combinatorial libraries by two hybrid methods.

In another embodiment, said TG2 inhibitor can be an inhibitor of TG2 gene expression.

An “inhibitor of gene expression” refers to a natural or synthetic compound that has a biological effect to inhibit or significantly reduce the expression of a gene. Consequently an “inhibitor of TG2 gene expression” refers to a natural or synthetic compound that has a biological effect to inhibit or significantly reduce the expression of the gene encoding the TG2 protein.

Inhibitors of TG2 gene expression for use in the present invention may be based on anti-sense oligonucleotide constructs. Anti-sense oligonucleotides, including anti-sense RNA molecules and anti-sense DNA molecules, would act to directly block the translation of TG2 mRNA by binding thereto and thus preventing protein translation or increasing mRNA degradation, thus decreasing the level of TG2, and thus activity, in a cell. For example, antisense oligonucleotides of at least about 15 bases and complementary to unique regions of the mRNA transcript sequence encoding the TG2 protein can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of TG2 gene expression for use in the present invention. TG2 gene expression can be reduced by contacting a subject or cell with a small double stranded RNA (dsRNA), or a vector or construct causing the production of a small double stranded RNA, such that TG2 expression is specifically inhibited (i.e. RNA interference or RNAi). Methods for selecting an appropriate dsRNA or dsRNA-encoding vector are well known in the art for genes whose sequence is known. shRNAs (short hairpin RNA) can also function as inhibitors of TG2 gene expression for use in the present invention.

Ribozymes can also function as inhibitors of TG2 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of TG2 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable.

Both antisense oligonucleotides and ribozymes useful as inhibitors of TG2 gene expression can be prepared by known methods. These include techniques for chemical synthesis such as, e.g., by solid phase phosphorothioate chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a mean of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and preferably cells expressing TG2. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are well known in the art.

Preferred viruses for certain applications are the adenoviruses and adeno-associated (AAV) viruses, which are double-stranded DNA viruses that have already been approved for human use in gene therapy. Actually at least 12 different AAV serotypes (AAV 1 to 12) are known, each with different tissue tropisms. Recombinant AAV are derived from the dependent parvovirus AAV2. The adeno-associated virus type 1 to 12 can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion and most recombinant adenoviruses are extrachromosomal.

Other vectors include plasmid vectors. Plasmid vectors have been extensively described in the art and are well known to those of skill in the art. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript, pSIREN. Other plasmids are well known to those of ordinary skill in the art. Additionally, plasmids may be custom designed using restriction enzymes and ligation reactions to remove and add specific fragments of DNA. Plasmids may be delivered by a variety of parental, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the antisense oligonucleotide, siRNA, shRNA or ribozyme nucleic acid sequence is under the control of a heterologous regulatory region, e.g., a heterologous promoter.

In a particular embodiment, the TG2 inhibitor of the invention may be used in combination with immunosuppressive treatment for the treatment of RPGN.

Said immunosuppression treatment corresponds to classical immunosuppression treatment which generally consists of oral steroids in combination with oral cyclophosphamide or azathioprine.

### Methods for Treatment

A further object of the invention relates to methods and compositions for the prevention or treatment of a subject afflicted with or susceptible to be afflicted with rapidly progressive glomerulonephritis (RPGN).

In one embodiment, the invention relates to a method for preventing or treating RPGN comprising administering a subject in need thereof with a therapeutically effective amount of a TG2 inhibitor of the invention.

In another embodiment, the invention relates to said method for preventing or treating RPGN associated to a disease selected from the group of Goodpasture syndrome, Immune complex diseases such as systemic lupus erythematosus, acute proliferative glomerulonephritis, chronic infection with RPGN, Henoch-Schönlein purpura, and IgA nephropathy, Anti-neutrophil cytoplasmic antibodies (ANCA)-associated vasculitis such as Wegener granulomatosis, microscopic polyangiitis, or Churg-Strauss syndrome or Wegener granulomatosis.

In a particular embodiment, said TG2 inhibitor of the invention is an inhibitor of TG2 activity.

In another particular embodiment, said TG2 inhibitor of the invention is an inhibitor of TG2 gene expression.

The term “subject” denotes a mammal, such as a rodent, a feline, a canine, and a primate. Preferably, a subject according to the invention is a human.

Preferably, said inhibitor is administered in a therapeutically effective amount. By a “therapeutically effective amount” is meant a sufficient amount of the TG2 inhibitor of the invention to treat and/or to prevent RPGN at a reasonable benefit/risk ratio applicable to any medical treatment.

In a particular embodiment, the invention relates to a method for treating RPGN comprising administering a subject in need thereof with a therapeutically effective amount of a TG2 inhibitor of the invention in combination with classical immunosuppressive treatment, currently used for the treatment of crescentic glomerulonephritis.

The TG2 inhibitor may be administered in the form of a pharmaceutical composition, as defined below.

It will be understood that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Pharmaceutical Compositions

A further object of the invention relates to a pharmaceutical composition for preventing or treating RPGN, said composition comprising a TG2 inhibitor of the invention.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The TG2 inhibitor(s) of the invention may be combined with pharmaceutically acceptable excipients, and optionally sustained-release matrices, such as biodegradable polymers, to form therapeutic compositions.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The TG2 inhibitor of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active substances in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The TG2 inhibitor of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

The invention will be further illustrated by the following figures and examples. However, these examples and figures should not be interpreted in any way as limiting the scope of the present invention.

### FIGURES

FIG. 1: Elisa for quantitative determination of albumin in mouse urine. Urinary albumin excretion was measured using a specific ELISA assay for quantitative determination of albumin in mouse urine (CellTrend GmbH). Serum creatinine was quantified spectrophotometricallly using colorimetric methods. Data represent the mean±sem (n=5-7 per group). ** p<0.01

FIG. 2: Urea concentration in mouse serum. Serum urea concentration was antified spectrophotometricallly using colorimetric methods. Data represent the mean±sem (n=6-7 per group).

FIG. 3: The count of the crescentic glomeruli was performed by an independent examiner on 3 μm tick sections stained with Masson trichrome. The proportion of crescentic glomeruli was determined by examination of at least 100 glomeruli per section. Data represent the mean±sem (n=5-7 per group). ** p<0.01

FIG. 4: Distance of migration of differentiated podocytes within 20 h in the wound assay. A wound assay was used to assess podocyte migration. Differentiated cells were seeded in a 35 mm μ-Dish (Ibidi) forming two confluent monolayers separated by a silicon insert of 500 μm width. After 1 day, the silicon insert was removed and 100 μM of MDC (monodansylcadaverin, Sigma-Aldrich #D4008) diluted in DMSO, a TG2 inhibitor, was added or not. Cells were fixed 20 h later and the distance between the two monolayers was measured. *p<0.05 vs. vehicle alone (n=3 per condition).

FIG. 5: Outgrowth from isolated glomeruli. For studying the role of TG2 specifically in podocytes, previously generated floxed TG2 mice (Nanda et al., 2001) were bred with 2.5P-Cre transgenic mice (Moeller et al., 2003) to generate mice with a specific deletion of TG2 in podocytes. Migration/proliferation study was performed in primary cultured podocytes isolated either from C57BL/6 or 2.5P-CreTG2lox mice. The isolation of glomeruli was performed by two-step sieving of kidneys (100 and 40 μM) as described earlier (Schiwek et al., 2004). The outgrowth of podocytes started between days 2 and 3. At day 4, podocyte outgrowth area was quantified on 20 glomeruli per mouse using ImageJ software. ** p<0.001, ***p<0.0001 vs. PodoTG2 control mice.

### EXAMPLE

To study the role of TG2 in the development of inflammatory glomerular injury, anti-glomerular basement membrane (GBM) glomerulonephritis (GN) was induced in TG2 knock-out mice and in wild-type mice of the same genetic background. TG2 knock-out (KO) mice, generated in Gerry Melino's Laboratory (IDI-IRCCS Biochemistry Lab, Department of Experimental Medicine, University Tor Vergata, Rome, Italy) were bred in our animal facility, These mice were backcrossed more than 12 generation onto the C57BL/6J genetic background whereas age- and sex-matched wild-type C57BL/6J mice used as controls were purchased from the Charles River Laboratories (L'arbresle, France).

10-12 week old wild type (WT) and TG2 KO males were preimmunized by subcutaneous injection with 200 μl of normal sheep IgG (Sigma-Aldrich Corp., St. Quentin Fallavier, France) emulsified to 6 mg/ml in 50% complete Freund's adjuvant (Sigma). 6 days later (day 1), GN was induced by intravenous injection of 50 μl sheep anti-GBM serum (nephrotoxic serum, NTS) under light anesthesia (isoflurane 2%). Serum injections were repeated daily on days 2 and 3. On day 4, 17 and 30, mice were sacrificed after body weight measurement and 12 hours urines collection with metabolic cages. Blood was obtained from mice by aortic catheterisation. Kidneys from each mouse were processed for immunohistochemistry. Serum and urines were stored at −80° C. until analysis.

TG2 Deficiency Protects Mice from Glomerular Injury.

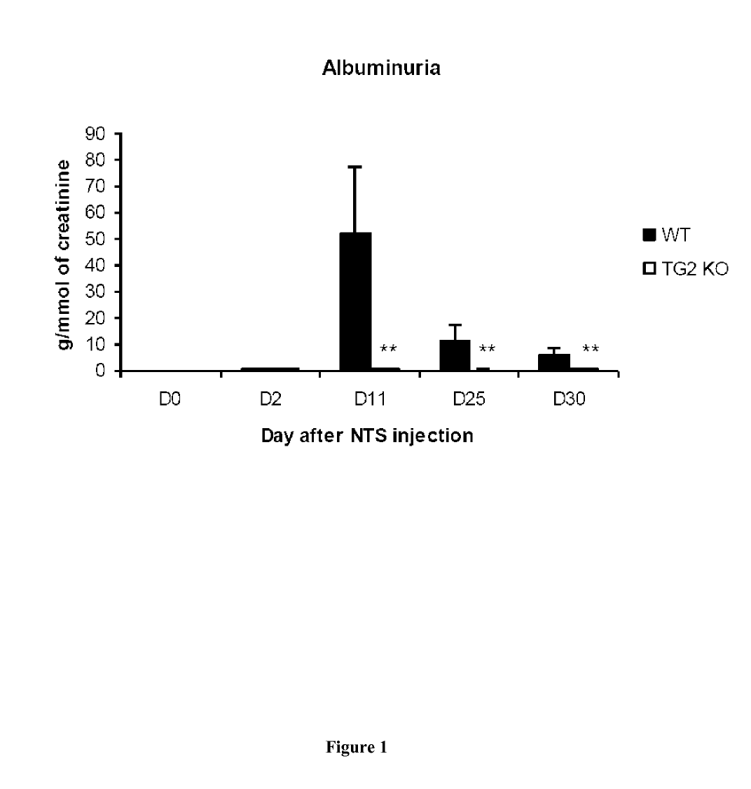

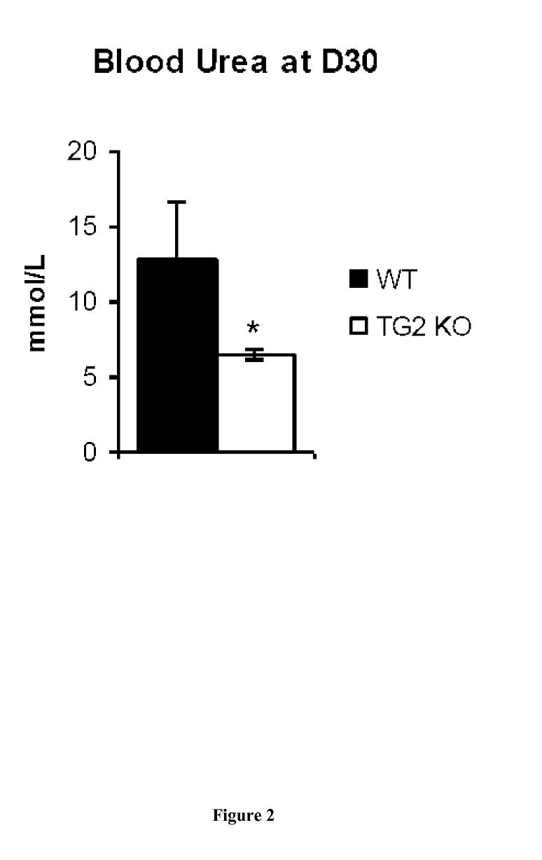

TG2 deficiency was associated with a delayed and attenuated glomerular injury, at the morphological and functional levels. Experimental GN provoked marked albuminuria in WT animals, compared to undetectable levels in TG2 KO mice during the progression of the disease, suggesting a strong contribution of TG2 to glomerular injury (FIG. 1). In line with this result, serum urea concentrations indicate a more pronounced renal failure in treated WT mice, compared with treated TG2 KO animals on day 30 (12.7±3.9 vs. 6.5±0.3 mmol/L) (FIG. 2).

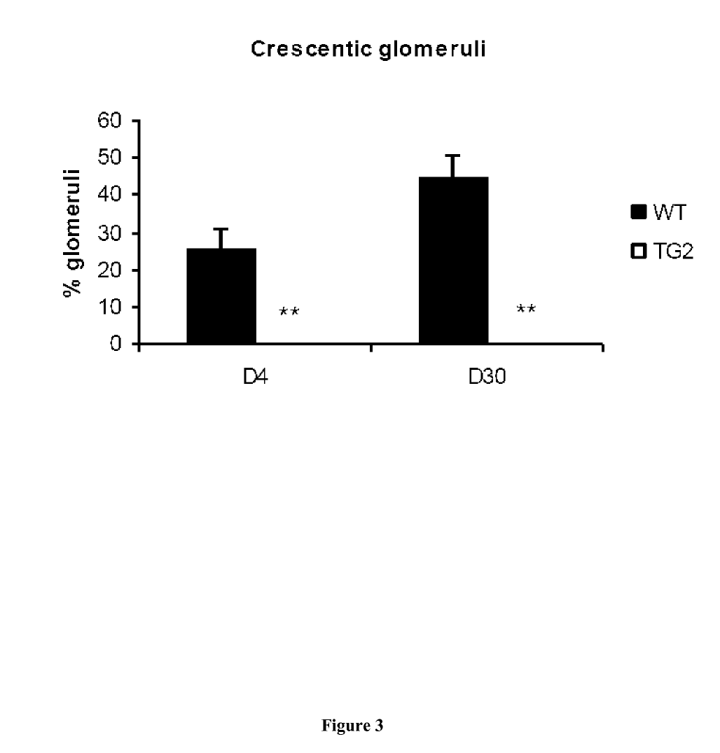

According to this result, histological analysis revealed that TG2 KO mice do not display any crescentic glomeruli, neither on day 4 during the acute renal aggression, nor on day 30 during the chronic progression of the disease (FIG. 3). In contrast, 25% of the glomeruli displayed crescents in WT animals at day 4; and this proportion rose to 45% at day 30, consistent with the progression of the renal failure.

Preliminary assessment of the inflammatory cells infiltration by immunohistochemistry revealed a strong decrease in the number of T-cells and monocytes/macrophages in the treated-TG2 kidneys as compared with treated-wild type kidneys.

TG2 is Expressed in Mouse and Human Podocytes.

As RPGN is characterized by proliferation and migration of podocytes in the urinary space, we next assessed the expression and function of TG2 specifically in podocytes. Results show that podocytes express TG2 at baseline and upon induction of experimental RPGN. Similarly, immunoreactive TG2 expression was markedly increased in various biopsies from kidneys with diagnosed RPGN with no expression in podocytes in other glomerular diseases such as minimal change disease and focal and segmental glomerulosclerosis (FSGS).

TG2 Induces a Migratory and Proliferative Phenotype in Podocytes In Vitro.

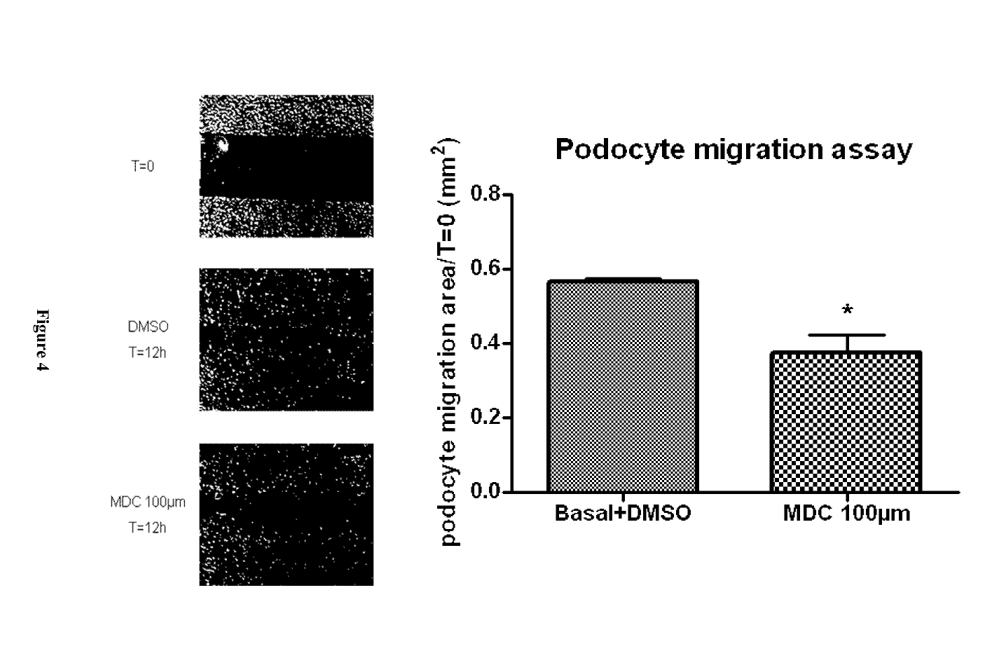

In vivo podocytes are terminally differentiated and stationary cells. During crescent formation in anti-GBM GN in mice, however, podocytes assume a migratory phenotype, attach with their apical membrane onto the parietal basement membrane and start to proliferate. Recent data confirm that podocytes contribute to crescents in humans, too. Migration of primary culture of mouse podocytes was significantly blunted by the TG2 inhibitor monodansylcadaverine (FIG. 4).

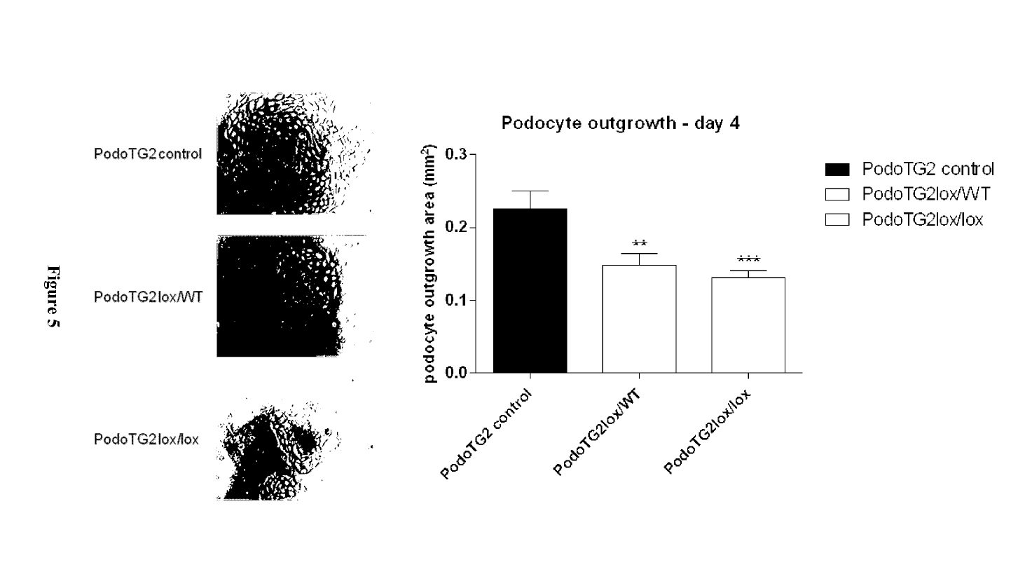

As an assay for podocyte crescent formation, we measured outgrowth epithelial cells from isolated decapsulated glomeruli from TG2−/− and TG2+/+ mice. The area of podocyte outgrowth strongly depended on the presence of a TG2 functional allele (0.86±0.18 mm2 vs. 1.61±0.21 mm2, P<0.05 TG2 (−/−) vs. TG2 (+/+)). Furthermore, outgrowths of podocytes from Podocin-Cre×TG2 lox/lox or Podocin-Cre TG2 lox/wt mice were also markedly blunted as compared with podocytes from Podocin-Cre×TG2 wt/wt mice (p<0.0001 and p<0.001 respectively), demonstrating the crucial role of TG2 in podocyte dedifferentiation (FIG. 5).

These data confers TG2 with a new, immunostimulatory role and pathophysiological role governing podocyte functions and that neutralization of TG2 activity or of its signalling partners could be of therapeutic use in glomerulonephritis.

Specific neutralization of TG2 is expected to be a therapeutic tool in auto-immune renal diseases. Meanwhile, we cannot exclude other role of TG2 in mediating the renal response to anti-GBM infusion or involvement of TG2 in alloimmune response, either during the alloimmunization process or during the effector response.

### CONCLUSION

TG2 expression was found to be markedly expressed in crescentic lesions in mice and human glomerulonephritic subjects. TG2 is mandatory to experimental RPGN, in part through promotion of proliferation and migration of podocytes. This study suggests that targeting the TG2 pathway would be clinically beneficial for treatment of severe RPGNs.

### REFERENCES

Throughout this application, various references describe the state of the art to which this invention pertains. The disclosures of these references are hereby incorporated by reference into the present disclosure.
Aeschlimann D, & Paulsson M. (1994). Transglutaminases: protein cross-linking enzymes in tissues and body fluids. Thromb Haemost 71(4), 402-415.
Case A, Ni J, Yeh L-A, Stein R L. (2005) Development of a mechanism-based assay for tissue transglutaminase-results of a hight-throughput screen and discovery of inhibitors. Anal Biochem 338, 237-244.
De Macédo P, Marrano C, Keillor J W. (2000) A direct continuous spectrophotometric assay for transglutaminase activity. Anal Biochem 285(1):16-20.
De Macédo P, Marrano C, Keillor J W. (2002) Synthesis of dipeptide-bound epoxides and alpha,beta-unsaturated amides as potential irreversible transglutaminase inhibitors. Bioorg Med Chem 10(2):355-60.
Duval E, Case A, Stein R L, Cuny G D. (2005). Structure-activity relationship study of novel tissue transglutaminase inhibitors. Bioorg Med Chem Lett 15, 1885-1889.
Folk J E, & Cole P W. (1966). Identification of a functional cysteine essential for the activity of guinea pig liver transglutaminase. J Chem Biol 241(13), 3238-3240.
Freund K F, Doshi K P, Gaul S L, Claremon D A, Remy D C, Baldwin J J, Pitzenberger S M, Stern A M. (1994). Transglutaminase inhibition by 2-[(2-oxopropyl)thio]imidazolium derivatives: mechanism of factor XIIIa inactivation. Biochemistry 33(33):10109-19.
Hausch F, Halttunen T, Mäki M, Khosla C. (2003). Design, synthesis, and evaluation of gluten peptide analogs as selective inhibitors of human tissue transglutaminase. Chem Biol 10(3):225-31.
Jayasena S D. (1999) Aptamers: an emerging class of molecules that rival antibodies in diagnostics. Clin Chem 1999; 45(9):1628-50.
Lai T S, Slaughter T F, Peoples K A, Hettasch J M, Greenberg C S. (1998). Regulation of human tissue transglutaminase function by magnesium-nucleotide complexes. J Biol Chem 273(3), 1776-1781.
Lorand L, & Conrad S M. (1984). Transglutaminases. Mol Cell Biochem 58 (1-2), 9-35.
Maiuri L, Clacci C, Ricciardelli I, Vacca L, Raia V, Rispo A, Griffin M, Issekutz T, Quaratino S, Londei M. (2005). Unexpected role of surface transglutaminase type II in celiac disease. Gastroenterology. 129(5):1400-13.
Moeller M J, Sanden S K, Soofi A, Wiggins R C, Holzman L B. (2003). Podocyte-specific expression of cre recombinase in transgenic mice. Genesis 35(1):39-42.

Mohan K, Pinto D, Issekutz T B. (2003). Identification of tissue transglutaminase as a novel molecule involved in human CD8_T cell transendothelial migration. J Immunol 171:3179-3186.
Nanda N, Iismaa S E, Owens W A, Husain A, Mackay F, Graham R M. (2001). Targeted inactivation of Gh/tissue transglutaminase II. J Biol Chem 276(23):20673-8.
Pardin C, Gillet S M, Keillor J W. (2006). Synthesis and evaluation of peptidic irreversible inhibitors of tissue transglutaminase. Bioorg Med Chem 14(24):8379-85.
Pardin C, Pelletier J N, Lubell W D, Keillor J W. (2008). Cinnamoyl inhibitors of tissue transglutaminase. J Org Chem 73(15):5766-75.
Schiwek D, Endlich N, Holzman L, Holthofer H, Kriz W, Endlich K. (2004). Stable expression of nephrin and localization to cell-cell contacts in novel murine podocyte cell lines. Kidney Int 66(1):91-101.

1.-8. (canceled)

9. A method for preventing or treating rapidly progressive glomerulonephritis (RPGN) in a subject in need thereof, comprising the step of
administering to said subject a therapeutically effective amount of a tissue transglutaminase (TG2) inhibitor.

10. The method of claim 9, wherein said RPGN is associated with a disease selected from the group consisting of Goodpasture syndrome, immune complex diseases, and anti-neutrophil cytoplasmic antibodies (ANCA)-associated vasculitis.

11. The method of claim 10, wherein said immune complex disease is selected from systemic lupus erythematosus, acute proliferative glomerulonephritis, chronic infection with RPGN, Henoch-Schönlein purpura, and IgA nephropathy.

12. The method of claim 10, wherein said ANCA-associated vasculitis is selected from the group consisting of Wegener granulomatosis, microscopic polyangiitis, Churg-Strauss syndrome, and Wegener granulomatosis.

13. The method of claim 9, wherein said TG2 inhibitor is an inhibitor of TG2 activity.

14. The method of claim 13, wherein said inhibitor of TG2 activity is monodansylcadaverine.

15. The method of claim 9, wherein said TG2 inhibitor is an inhibitor of TG2 gene expression.

16. The method of claim 9, wherein said TG2 inhibitor is administered in combination with an immunosuppressive treatment.

17. A method for alleviating podocytes proliferation and/or migration in a subject afflicted with rapidly progressive glomerulonephritis (RPGN), comprising the step of
administering to said subject a therapeutically effective amount of a tissue transglutaminase (TG2) inhibitor.

18. A pharmaceutical composition for the prevention or treatment of RPGN, comprising
a TG2 inhibitor, and
a pharmaceutically acceptable excipient.

19. The pharmaceutical composition of claim 18, further comprising an immunosuppressive agent.
